(19) United States
(12) Patent Application Publication
(10) Pub. No.: US 2011/0258747 A1
(43) Pub. Date: Oct. 27, 2011

(54) CONTROL CIRCUIT FOR AUTOMATIC SHADING WELDING HELMET

(76) Inventor: Woon Su SEO, Gwangmyeong-si (KR)

(21) Appl. No.: 13/088,806

(22) Filed: Apr. 18, 2011

(30) Foreign Application Priority Data
Apr. 21, 2010 (KR) 10-2010-0036654

(51) Int. Cl. A61F 9/06

(52) U.S. Cl. 2/8.8

(57) ABSTRACT

A control circuit for an automatic shading welding helmet prevents a LCD driving from being delayed owing to a reset delay of a micro computer. The control circuit for the automatic shading welding helmet having a welding detecting part, a solar cell, a micro computer, a LCD driving part, a LCD driving power, a LCD part, a high voltage supplying part, and a power supply includes a welding detecting signal processing part for outputting a welding detecting signal when the welding detecting signal is inputted from the welding detecting part and allowing the power to be supplied to the micro computer through the power supply; and a temporary driving controller for allowing a temporary power to be directly supplied to the LCD driving part before a reset of the micro computer and allowing the power of the power supply to be supplied to the LCD driving part.

### CROSS REFERENCES

Applicant claims foreign priority under Paris Convention and 35 U.S.C. §119 to Korean Patent Application No. 10-2010-0036654, filed Apr. 21, 2010, with the Korean Intellectual Property Office, where the entire contents are incorporated herein by reference.

### BACKGROUND OF THE INVENTION

1. Field of the Invention

The present invention relates to a control circuit for an automatic shading welding helmet, and more particularly to a control circuit for an automatic shading welding helmet in that it can prevent a LCD driving from being delayed owing to a reset delay of a micro computer, whereby improving a reaction velocity on the initial welding light.

2. Description of the Prior Art

Generally, the welding is a technique of jointing same or different kinds of metal materials by partially applying a heat and a pressure to them at the same time. During the welding, since a strong light or broken pieces are generated, it can cause loss of vision of the worker or inflict an injury on an eyeball or a face thereof.

Accordingly, the work wears a welding helmet for protecting the eyes and faces thereof from the strong light or broken pieces generated during the welding. Recently, an automatic shading welding helmet for driving a shading lens having a LCD and blocking the light according to the detection of the arc light is widely used.

FIG. 1 is a block diagram illustrating a control circuit for a conventional automatic shading welding helmet.

Referring to FIG. 1, the conventional control circuit for the automatic shading welding helmet includes a solar cell 100, a power supply 110, an auto-on switch 120, a welding detecting part 130, a micro computer 140, a LCD driving power supply 150, a boost part 160, a high voltage controller 170, a LCD driving part 180, and a LCD part 190.

The solar cell 100 and the power supply 110 serve to supply a power to each circuit part. That is, the solar cell 100 serves to supply the power to each circuit part according to the switching operation of the auto-on switch 120 during initial operation thereof before welding start and completion of a reset of the micro computer 140 and the power supply 110 serves to supply the power to each circuit part after the completion of the reset of the micro computer 140.

The welding detecting part 130 serves to amplify a detected current proportion to a welding light intensity or a frequency signal generated during welding through an antenna 15, filter a noise except for the frequency according to the welding, and control a detecting sensitivity according to the welding light intensity.

The micro computer 140 serves to generate a control signal for LCD shading control when the welding is detected and the LCD driving power supply 150 serves to supply the power to the LCD driving part 180 according to the control signal of the micro computer 140. Recently, since there is a defect in that the micro computer of a button type manually controls the shading degree thereof, it uses a digital micro computer in that the shading degree can be automatically controlled and the LCD driving pulse can be automatically generated.

The boost part 160 serves to boost the power voltage (six times voltage; 36V) and the high voltage controller 170 serves to apply the instant high voltage, which is boosted in the boost part 160, to both ends of the LCD part 190.

The LCD driving part 180 drives LCD in a normal status after the LCD is initially driven by the high voltage controller 170.

In the conventional art, where the driving power is automatically supplied to the micro computer 140 through the solar cell 100 before welding, if the reset of the micro computer 140 is completed, the driving power is supplied from the power supply 110. However, there is a problem in that the reset time is increased owing to the initial driving power supplying of the solar cell 100 and the LCD driving is delayed owing to the increase of the reset time.

That is, the micro computer 140 uses much power for automatic shading control and LCD driving pulse generation. Meanwhile, since the power applied by the solar cell 100 is weak, it takes a long time to reset the micro computer. Also, because it is insufficient to apply the power of the solar cell in a dark place, the reset thereof becomes bad.

After all, where the reset of the micro computer is not completed, if the welding is performed, since the delay of the LCD driving is generated, the worker's eye is exposed to the welding light, thereby it can cause loss of vision of the worker.

### SUMMARY OF THE INVENTION

Accordingly, the present invention has been made to solve the above-mentioned problems occurring in the prior art, and an object of the present invention is to provide a control circuit for an automatic shading welding helmet in that, when an automatic shading function is conducted through a welding detection, the shading function is controlled through a temporary driving part before a reset of a micro computer and is controlled through the micro computer after the reset of the micro computer is completed, thereby it can prevent the LCD driving from being delayed owing to a reset delay of the micro computer.

To accomplish the object, the present invention provides a control circuit for an automatic shading welding helmet comprising: a welding detecting part for detecting a start of a welding; a solar cell for supplying a power to the welding detecting part; a micro computer for performing a shading control operation when the welding is detected; a LCD driving part for generating a driving pulse corresponding to the shading control signal of the micro computer; a LCD driving power supply for supplying a driving power to the LCD driving part according to the shading control signal of the micro computer; a LCD part for shutting out a welding light through a LCD array corresponding to a driving signal of the LCD driving part; a high voltage supplying part for supplying an instant high voltage to the LCD part and preventing the high voltage supplied to the LCD part from being supplied to the LCD driving part; a power supply for supplying a power to each circuit part; a welding detecting signal processing part for outputting a welding detecting signal when the welding detecting signal is inputted from the welding detecting part and allowing the power to be supplied to the micro computer through the power supply; and a temporary driving controller for allowing a temporary power to be directly supplied to the LCD driving part before a reset of the micro computer according to the welding detecting signal of the welding detecting signal processing part and allowing the power of the power supply to be supplied to the LCD driving part under the control of the micro computer after the reset of the micro computer.

Preferably, the control circuit for an automatic shading welding helmet further comprising a first switch for connecting and disconnecting the power supplied to the micro computer and the LCD driving power supply in the power supply according to the output of the welding detecting signal processing part, which is formed between the welding detecting signal processing part and the micro computer.

Preferably, the temporary driving controller comprises: a temporary driving signal generating part for generating a temporary driving signal when the welding detecting signal is inputted; and a second switch for allowing the power of the power supply to be directly supplied to the LCD driving part under the control of the temporary driving signal generating part before the reset of the micro computer and allowing the power of the power supply to be supplied to the LCD driving part under the control of the micro computer after the reset of the micro computer.

### BRIEF DESCRIPTION OF THE DRAWINGS

The above as well as the other objects, features and advantages of the present invention will be more apparent from the following detailed description taken in conjunction with the accompanying drawings, in which:

FIG. 1 is a block diagram illustrating a control circuit for a conventional automatic shading welding helmet; and

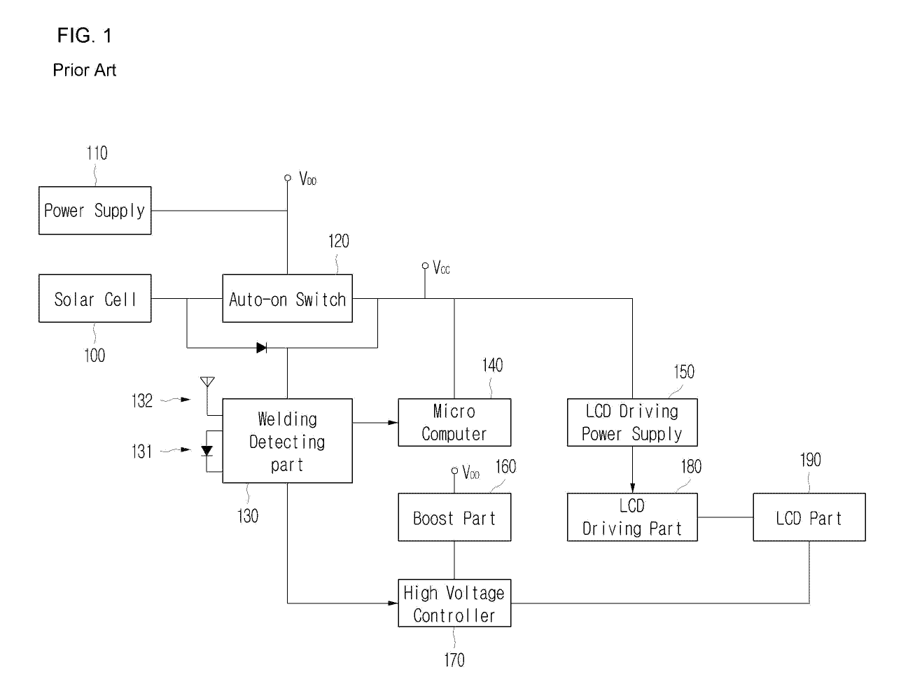

FIG. 2 is a block diagram illustrating a control circuit for an automatic shading welding helmet according to the present invention.

### DETAILED DESCRIPTION OF THE EXEMPLARY EMBODIMENTS

Hereinafter, preferred embodiments of the present invention will be described with reference to the accompanying drawings.

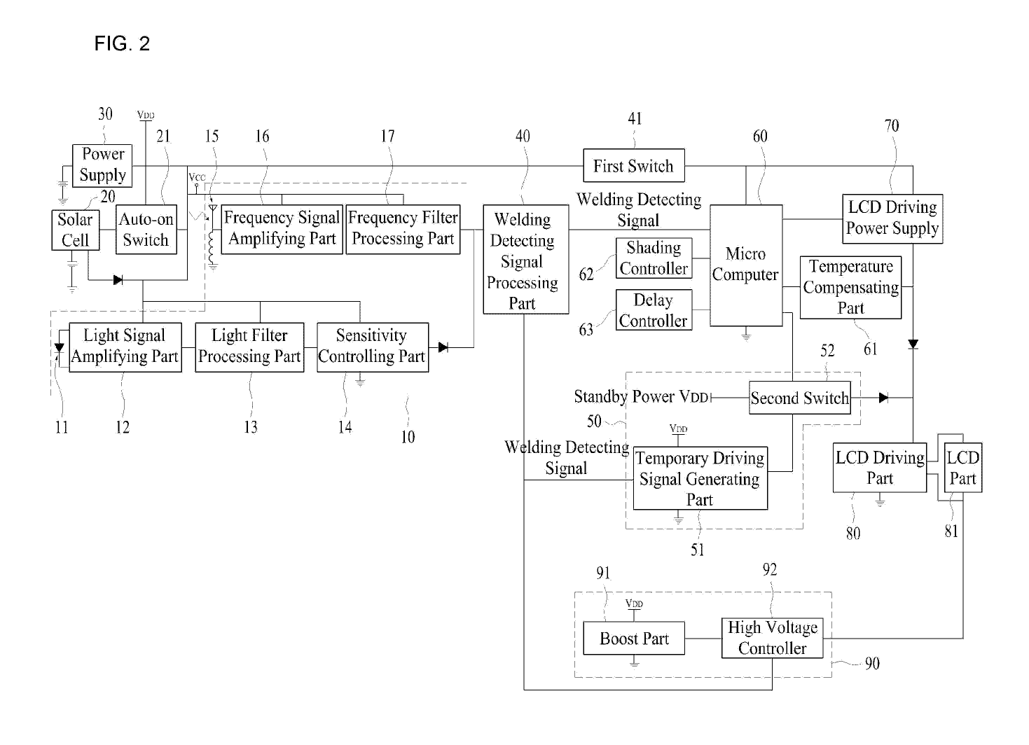

FIG. 2 is a block diagram illustrating a control circuit for an automatic shading welding helmet according to the present invention.

Referring to FIG. 2, the control circuit for an automatic shading welding helmet according to the present invention includes a welding detecting part 10, a solar cell 20, a power supply 30, a welding detecting signal processing part 40, a temporary driving controller 50, a micro computer 60, a LCD driving power supply 70, a LCD driving part 80, a LCD part 81, and a high voltage supplying part 90.

The welding detecting part 10 for detecting a start of a welding includes a light signal amplifying part 12, a light filter processing part 13, a sensitivity controlling part 14, a frequency signal amplifying part 16, and a frequency filter processing part 17.

The light signal amplifying part 12 serves to amplify a detected current signal when the detected current proportion to a welding light intensity is inputted from a photo sensor 11. Also, the light filter processing part 13 serves to filter a light of the sun or a peripheral lighting etc. except for the welding light in the amplified signal of the light signal amplifying part 12 and the sensitivity controlling part 14 serves to control a sensitivity thereof according to the welding light intensity.

Moreover, the frequency signal amplifying part 16 serves to amplify a frequency signal generated during welding through an antenna 15 to output a stable signal and the frequency filter processing part 17 serves to filter a noise except for the frequency according to the welding in the outputted signal of the frequency signal amplifying part 16.

The solar cell 20 and the power supply 30 serves to supply a power to each circuit part. That is, the solar cell 20 serves to supply a VCC power to each circuit part of a welding detecting part 10 in standby status and the power supply 30 serves to automatically turn on when the welding is detected and supply the power to each circuit part including of the micro computer 60.

An auto-on switch 21 for connecting and disconnecting the power so as to selectively supply the power to each circuit part through the solar cell 20 or the power supply 30 is connected to a rear end of the solar cell 20 or the power supply 30. Here, auto-on switch 21 is switched off in standby status in order that the solar cell 20 supplies the VCC power to each circuit part of the welding detecting part 10 and is switched on after the detection of the welding in order that the power supply 30 supply a VDD power to each circuit part of the welding detecting part 10.

The welding detecting signal processing part 40 allows the VDD power to be supplied to the micro computer 60 through the power supply 30 when the welding is detected, allows the LCD part 81 to be driven through a temporal driving power, and allows an instant high voltage to be applied to both ends of the LCD part 81 through a high voltage controller 92.

The welding detecting signal processing part 40 includes a first switch 41 for connecting and disconnecting the VDD power supplied to the micro computer 60 and the LCD driving power supply 70 in the power supply 30 according to the output of the welding detecting signal processing part 40, which is connected to input terminals of the micro computer 60 and the LCD driving power supply 70.

The micro computer 60 serves to perform a shading control operation when the welding is detected. That is, the micro computer 60 serves to perform a reset operation for the shading control by receiving the VDD power from the power supply 30 when the welding detecting signal is inputted through the welding detecting signal processing part 40 and output a shading control signal to the LCD driving part 80 after the reset operation.

At this time, the micro computer 60 is connected to a temperature compensating part 61, a shading controller 62, and a delay controller 63 so as to maintain a constant shading degree irrespective of a peripheral temperature change and set an output time of the control signal during detection of the welding.

The LCD driving power supply 70 serves to supply the VDD power of the power supply 30 to the LCD driving part 80 according to the control signal of the micro computer 60. Also, the LCD driving part 80 serves to generate a driving pulse corresponding to the control signal of the micro computer 60 and supply the power to both ends of the LCD part 81 and the LCD part 81 serves to shut out the welding light through a LCD array corresponding to the driving signal of the LCD driving part 80.

The high voltage supplying part 90 serves to improve a shading speed by supplying the instant high voltage to the LCD part 81 during initial driving when the welding detecting signal is applied. The high voltage supplying part 90 includes a boost part 91 and the high voltage controller 92. The boost part 91 serves to boost the power voltage and the high voltage controller 92 serves to apply the instant high voltage, which is boosted in the boost part 91, to both ends of the LCD part 81 through one pulse.

The temporary driving controller 50 serves to control a driving of the LCD driving part 80 according to a completion or incompletion of the reset of the micro computer 60 when the welding detecting signal is inputted through the welding detecting signal processing part 40.

At this time, the temporary driving controller 50 includes a temporary driving signal generating part 51 for generating a temporary driving signal when the welding is detected and a second switch for supplying the power to the LCD driving part 80 under the control of the micro computer 60 or the power to the LCD driving part 80 under the control of the temporary driving signal generating part 51.

According to this construction, when the reset of the micro computer 60 is not completed, the second switch 52 is switched on, so that the VDD power of the power supply 30 is supplied to the LCD driving part 80 in standby status according to the control of the temporary driving signal generating part 51. In the meantime, when the reset of the micro computer 60 is completed, the second switch 52 is switched off, so that the VDD power of the LCD driving power supply 70 is supplied to the LCD driving part 80.

Hereinafter, the operation of The control circuit for an automatic shading welding helmet according to the present invention will be described with reference to FIG. 2. Here, the light is detected by the photo sensor.

First, the auto-on switch 21 is switched off in standby status, so that the solar cell 20 supplies the VCC power to each circuit part of the welding detecting part 10.

In this state, where the welding is generated, the detected current proportion to the welding light intensity is generated in the photo sensor 11. Then, the light signal amplifying part 12 amplifies the detected current signal, the light filter processing part 13 filters the light of the sun or the peripheral lighting etc. except for the welding light in the amplified signal of the light signal amplifying part 12, and the sensitivity controlling part 14 controls the sensitivity thereof according to the welding light intensity. At this time, the power supply 30 maintains the standby status. When the auto-on switch 21 is switched on according to the detection of the welding light, it supplies the VDD power to the power supply 30 and each circuit part.

Continuously, the welding detecting signal processing part 40 detects the welding light generating signal from the output signal of the sensitivity controlling part 14, so that the first switch 41 is switched on, thereby the VDD power is supplied to the micro computer 60 and the LCD driving power supply 70 and the welding detecting signal is applied to the micro computer 60, the temporary driving signal generating part 51, and the high voltage controller 92.

Thus, the high voltage controller 92 applies the instant high voltage, which is boosted in the boost part 91, to both ends of the LCD part 81 through one pulse, thereby starting the initial driving of the LCD part 81.

Then, the temporary driving signal generating part 51 generates the temporary driving signal according to the welding detecting signal, so that the second switch 52 is switched on, thereby the VDD power of the standby status is supplied to the LCD driving part 80 and the LCD driving part 80 drives the LCD part 81 according to the control signal of the temporary driving signal generating part 51.

Thereafter, when the reset of the micro computer 60 is completed, the second switch 52 is switched off, so that the control signal for shading control is applied to the VDD power of the LCD driving power supply 70. Accordingly, the LCD driving part 80 is driven through the power supplied from the LCD driving power supply 70.

While this invention has been described in connection with what are presently considered to be the most practical and preferred embodiments, it is to be understood that the invention is not limited to the disclosed embodiments and the drawings, but, on the contrary, it is intended to cover various modifications and variations within the spirit and scope of the appended claims.

What is claimed is:

1. A control circuit for an automatic shading welding helmet comprising:
a welding detecting part for detecting a start of a welding;
a solar cell for supplying a power to the welding detecting part;
a micro computer for performing a shading control operation when the welding is detected;
a LCD driving part for generating a driving pulse corresponding to the shading control signal of the micro computer;
a LCD driving power supply for supplying a driving power to the LCD driving part according to the shading control signal of the micro computer;
a LCD part for shutting out a welding light through a LCD array corresponding to a driving signal of the LCD driving part;
a high voltage supplying part for supplying an instant high voltage to the LCD part and preventing the high voltage supplied to the LCD part from being supplied to the LCD driving part;
a power supply for supplying a power to each circuit part;
a welding detecting signal processing part for outputting a welding detecting signal when the welding detecting signal is inputted from the welding detecting part and allowing the power to be supplied to the micro computer through the power supply; and
a temporary driving controller for allowing a temporary power to be directly supplied to the LCD driving part before a reset of the micro computer according to the welding detecting signal of the welding detecting signal processing part and allowing the power of the power supply to be supplied to the LCD driving part under the control of the micro computer after the reset of the micro computer.

2. A control circuit for an automatic shading welding helmet as claimed in claim 1, further comprising a first switch for connecting and disconnecting the power supplied to the micro computer and the LCD driving power supply in the power supply according to the output of the welding detecting signal processing part, which is formed between the welding detecting signal processing part and the micro computer.

3. A control circuit for an automatic shading welding helmet as claimed in claim 1, wherein the temporary driving controller comprises;
a temporary driving signal generating part for generating a temporary driving signal when the welding detecting signal is inputted; and
a second switch for allowing the power of the power supply to be directly supplied to the LCD driving part under the control of the temporary driving signal generating part before the reset of the micro computer and allowing the power of the power supply to be supplied to the LCD driving part under the control of the micro computer after the reset of the micro computer.
